(19) United States
(12) Patent Application Publication
(10) Pub. No.: US 2022/0125930 A1
(43) Pub. Date: Apr. 28, 2022

(54) SYSTEM AND METHOD FOR FABRICATION OF LARGE, POROUS DRUG-SILK MATERIALS USING CRYOGRANULATION

(71) Applicant: Trustees of Tufts College, Medford, MA (US)

(72) Inventors: David L. Kaplan, Concord, MA (US); Ilia A. Rodionov, Medford, MA (US)

(21) Appl. No.: 17/310,586

(22) PCT Filed: Feb. 12, 2020
(86) PCT No.: PCT/US2020/018006

§ 371 Date: Aug. 12, 2021

Related U.S. Application Data
(60) Provisional application No. 62/804,406, filed on Feb. 12, 2019.

(51) Int. Cl. A61K 47/42; A61K 9/48; A61K 45/06

(52) U.S. Cl. CPC A61K 47/42; A61K 9/4825; A61K 45/06; A61K 9/4833

(57) ABSTRACT

A method of making drug-eluting regenerated silk fibroin particles using cryogranulation. The method has a first step of injecting a mixture into a super-cooled fluid, the mixture including regenerated silk fibroin and at least one medicament. A second step of incubating the drug-eluting particles in the super-cooled fluid to promote cryogelation may also be used. The size distribution, morphology, and cross-linking efficiency of the particles can depend on several controllable variable, such as starting concentrations of cross-linking agents and silk fibroin in the mixture, the injection pressure, and the temperature of the super-cooled fluid.

### CROSS-REFERENCE TO RELATED APPLICATIONS

This application is based on, claims priority to, and incorporates herein by reference in its entirety, U.S. Provisional Patent Application Ser. No. 62/804,406, filed on Feb. 12, 2019 and entitled “System and Method for Fabrication of Large, Porous Drug-Eluting Silk Materials Using Cryogranulation”.

### STATEMENT REGARDING FEDERALLY SPONSORED RESEARCH

This invention was made with government support under grant number FA9550-17-1-0333 awarded by the United States Air Force and grant numbers NS094218 and AR070975 awarded by the National Institutes of Health. The government has certain rights in the invention.

### BACKGROUND

Regenerated silk fibroin (RSF) is a protein biopolymer with biocompatibility, aqueous processability, and useful mechanical characteristics for biomaterials in the forms of films, hydrogels, sponges, fibers, and particles. RSF-based particles (RSFPs) with tunable morphologies and physical-chemical properties are useful as drug delivery systems (DDSs). For example, RSFPs less than 200 nm in diameter can be used as intravenous DDSs with extended half-lives in circulation, for controlled pharmacokinetics. Particles several microns in diameter can be used subcutaneously and intravenously to localize at injection sites. Large biopolymer-based particles with diameters 50 to 1,000 μm have a broad range of potential applications in photonic materials, as chromatography column solid supports for separations, in field-responsive rheological fluids, and as injectable DDSs for chemotherapeutic treatment of tumors and bacterial infections.

Spherical and cubical meso-particles for drug release are conventionally produced from gelatin, polyvinyl alcohol, polyacrylamide, and silicone. Polyacrylic and polyvinylalcohol-co-acrylamide spheres, 100 to 800 μm in dimeter, have been produced by double emulsion polymerization. Microfluidic coagulation and mechanical grinding of PVA-sheets have been shown to produce drug eluting polyvinyl alcohol-based particles of about 180-900 μm in diameter. A similar coagulation approach has been successfully applied to fabricate silk sericin/lignin blended beads up to 2,000 μm in diameter for the removal of Cr(IV) from waste water.

Silk-based materials bind doxorubicin (DoxR), with beta-sheet content of the SF controlling release kinetics. Silk-based micro- and meso-spheres has been explored, including those generated by salting-out, self-assembly, spray-drying, and mechanical grinding. Microfluidic techniques using laminar flow encapsulation and electrospinning offered effective control over the particle size over the range of 100-2,000 μm. The bulk emulsification-based assembly of silk particles by a drop dissolution techniques have produced spheres with monolith internal morphology. However, continuous production of polymeric meso-particles by microfluidic approaches using a double emulsion processes require precise tuning of the injector nozzle diameter and control over the rheology and fluid dynamics of the injected polymer solutions. In addition, such techniques require careful selection of the starting monomer-to-initiator ratio, stirring rate, temperature and pH of the emulsification bath.

Faster granulation techniques based on high shear phase separation of liquefied RSF in supercritical CO₂ produced monodisperse silk nano-particles have also been studied and are suggestive of an alternative for the assembly of silk. However, there is a large expense of scale-up when using CO₂-assisted setups for high shear emulsification of silk solutions.

Thus, what is needed are novel, simplified systems and methods for quickly forming regenerated silk fibroin particles that may be useful for drug delivery systems.

### SUMMARY OF THE DISCLOSURE

Systems and methods described herein relate to, inter alia, production of porous drug-eluting particles using cryogranulation. Consequently, the present disclosure addresses the aforementioned drawbacks by providing novel systems and methods for the fabrication of large sized silk particles using cryogelation. These advancements are useful for many applications, including drug delivery. The size distribution, morphology, and cross-linking efficiency of the resulting RSFPs depends upon the concentration of a cross-linking agent, a silk solution concentration, a coagulation bath temperature, and an injection pressure.

In one aspect, the present disclosure provides a method of making drug-eluting particles. The method can comprise injecting a mixture into a super-cooled fluid, the mixture comprising regenerated silk fibroin and at least one medicament, wherein the super-cooled fluid has a temperature of less than −40 degrees Celsius. The super-cooled fluid can be an organic solvent, organic mixture, or organic solution.

In another aspect, the present disclosure provides a method of making drug-eluting particles. The method can comprise injecting a mixture into a super-cooled fluid, the mixture comprising regenerated silk fibroin and at least one medicament, wherein the super-cooled fluid consists essentially of butane, pentane, hexane, heptane, octane, nonane, decane, or a mixture thereof.

In one aspect, the present disclosure provides a method of treating a subject. The method can comprise administering a drug-eluting particles made by one of the methods described herein to the subject.

In another aspect, the present disclosure provides a regenerated silk fibroin particle for drug elution. The regenerated silk fibroin particle may be characterized by being formed by one of the methods provided herein.

In another aspect, the present disclosure provides a system for making drug-eluting particles. The system may include a coagulation container system configured to maintain a temperature below −40 degrees Celsius for the fluid retained therein; a super-cooled fluid located within the coagulation container system; a source of a mixture comprising at least one medicament and regenerated silk fibroin, the mixture optionally comprising a cross-linking agent; and an injection unit positioned above a surface of the super-cooled fluid and configured to inject the mixture into the super-cooled fluid.

In one aspect, the present disclosure provides a system for making drug-eluting particles. The system can comprise one or more components described herein for the automatic execution of any of the method steps described herein.

These and other advantages and features of the present invention will become more apparent from the following detailed description of the preferred aspects of the present invention when viewed in conjunction with the accompanying drawings. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention. It should be understood, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings described herein are for illustration purposes only and are not intended to limit the scope of the disclosed embodiments in any way. The drawings described herein are for illustrative purposes only of selected embodiments and not all possible implementations, and are not intended to limit the scope of any of the various embodiments. It is understood that the drawings are not drawn to scale.

FIG. 1 illustrates a process flowchart of a method of making drug-eluting particles, in accordance with one aspect of the present disclosure.

FIG. 2 illustrates a process flowchart of a method of treating a subject, in accordance with another aspect of the present disclosure.

FIG. 3 illustrates a depiction of a regenerated silk fibroin particle for drug elution, in accordance with one aspect of the present disclosure. The interior composition of the RSFP has been represented using multiple symbols or elements, which are not depicted to scale.

FIG. 4 illustrates a system for making drug-eluting particles, in accordance with another aspect of the present disclosure.

FIG. 5 illustrates a depiction of the apparatus of the experiment of Example 1.

FIG. 6 illustrates measured rheological properties of the RSF/cross-linker mixtures for cryo-granulation in the experiment of Example 1; density of each batch was assessed gravimetrically; shear viscosity estimated based on the readings obtained on the rotational viscometer at a fixed 15% deformation and at a frequency range between 0.1-100 rad/s.

FIG. 7 panel (A) illustrates measured ATR-FTIR spectra of the experiment of Example 1 for: freeze-dried silk fibroin before (A, 1) and after cryogelation triggered by acetone (A, 2), EGDE (A, 3), or PPGDE (A, 5). FTIR spectra of chemically cross-linked RSF for EGDE (A, 4) and PPGDE (A, 6). Panel (B) shows the DSC characterizations of the experimental silk microspheres at −10 to 350° C. Panel (C) shows the DSC characterizations of the experimental silk microspheres at 160 to 245° C.

FIG. 8 illustrates estimations of mean RSFPs size by fluorescent microscopy of the water-swollen cryo-RSFPs in the experiment of Example 1; images were taken under GFP filter, 10× magnification. The images were contrasted and converted to binary offsets, counted and the mean particle Feret diameter frequencies were estimated with ImageJ software (ellipticity preset to 0.0-1.0).

FIG. 9 panels (a), (b), (c), and (d) all illustrate number average distribution profiles of RSFPs by Feret diameter for the experiment of Example 1. Each plotted value was the mean result of three microscopic readings and calculations. Each particulate sample was fabricated in duplicate.

FIG. 10 panels (A), (B), (C), and (D) all illustrate compilations of experimental SEM images for the RSFPs in the experiment of Example 1. The RSFPs were fabricated at different combinations of parameters including cross-linker type/starting mass balance/injection pressure. Each micrograph shows an 80× magnification of the particles general view and contains a 300× inserts of each batch typical surface topology. The leftmost column of each section contains 1500-200× magnification of the RSFPs microporous inner morphology.

FIG. 11 illustrates experimental z-stacked confocal imaging of the morphology and distribution of RPTEC/TERT1 (panels (A)-(D)) and HNF (panels (E)-(F)) cells within the macro-scale silk-based cryogels labeled with Alexa Fluor 546; the following cross-linkers were used to prepare the cryogel scaffolds: 30 mmol/g EGDE (panels (A), (E)), 30 mmol/g PPGDE (panels (B), (F)), 20% vol. acetone (panel (C)), and 15% wt. PEG-400 (panel (D). The scale bars on the micrographs are 50 μm each.

FIG. 12 panel A illustrates experimental release properties of the acetone-cross-linked RSFPs pre-laden with the doxorubicin chloride and antibiotics that were evaluated photometrically and via the disc-diffusion method, respectively. Panel B depicts an assessment of the S. aureus XEN29 disc-diffusion inhibition zone via bioluminescent imaging at 48 hours post application. Numerical captions above the samples relate to the concentration of drug (mg/mL) in the starting RSF/antibiotic mixtures.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices and methods relating to RSF-based particles are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, the term “super-cooled fluid” has its ordinary meaning in the art. In one some aspects, a “super-cooled fluid” defines a fluid, such as a liquid or a gas, that has a temperature below its freezing point, yet it maintains fluid state and does not solidify.

As used herein, the term “biocompatible” refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo.

As used herein, the term “macroparticle” refers to a particle having a diameter of at least 1 millimeter. In some embodiments, a population of particles is considered a population of macroparticles if the mean diameter of the population is equal to or greater than 1 millimeter.

As used herein, the term “microparticle” refers to a particle having a diameter between 1 micrometer and 1 millimeter. In some embodiments, a population of particles is considered a population of microparticles if the mean diameter of the population is between 1 micrometer and 1 millimeter.

As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, a population of particles is considered a population of nanoparticles if the mean diameter of the population is equal to or less than 1000 nm.

As used herein, a material, additive, and/or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Described herein are systems and methods related to the production of drug-eluting regenerated silk fibroin particles using cryogranulation. The simplified method of fabrication is capable of creating drug-eluting silk particles having various sizes and porosities. The produced particles have many practical uses, such as in chromatography column solid supports for separations, in field-responsive rheological fluids, and as injectable DDSs for treatment of a subject, such as chemotherapeutic treatment of tumors and bacterial infections.

### Methods

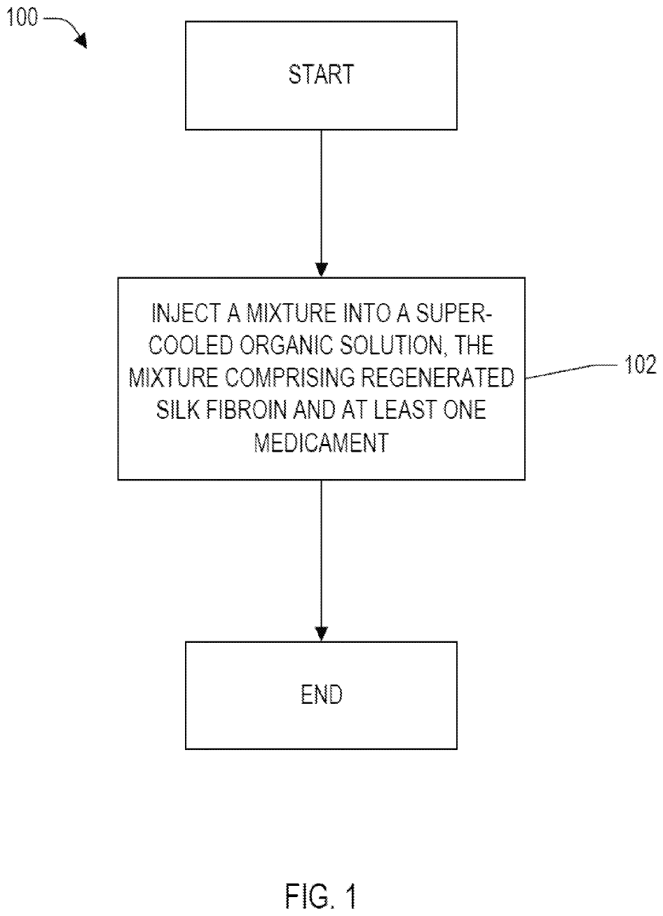

FIG. 1 depicts a method 100 of making drug-eluting particles. The method comprises a first step 102 of injecting a mixture into a super-cooled fluid, the mixture comprising regenerated silk fibroin and at least one medicament.

Although not depicted, the method 100 may further comprise the step of incubating the drug eluting particles in the super-cooled fluid to promote cryogelation. This incubation may occur for about 5, 10, 15, 20, 25, 30, or 60 minutes. The super-cooled fluid incubation may specifically occur for between 5 and 15 minutes. After this incubation period, the method 100 may further comprise a step of extracting the drug eluting particles from the super-cooled fluid. A basket or liquid-permeable membrane may be positioned in the super-cooled fluid and be used to extract the drug-eluting particles. The rapid formation of particles allows for the above steps to be incorporated into a batch, semi-batch, or continuous production process. The super-cooled fluid can have a temperature of less than −40 degrees Celsius, less than −50 degrees Celsius, less than −55 degrees Celsius, less than −60 degrees Celsius, less than −65 degrees Celsius, or less than −70 degrees Celsius.

After extraction, the method 100 may comprise a step of further incubating the drug-eluting particles in a cooling chamber in order to facilitate internal cryogelation of the silk fibroin. Residual super-cooled fluid may also be removed during this step. The cooling chamber may have a temperature of about −10, −15, −20, −30, −35, or −40 degrees Celsius. The temperature may be specifically maintained around −20 degrees Celsius. The incubation in the cooling chamber may be for a period of at least 10 hours, 20 hours, 30 hours, or longer. The method may further comprise a step of lyophilizing the drug eluting particles, which may take place after the cooling chamber incubation. The lyophilization may occur at a pressure below 0.1, 0.05, 0.01, 0.05, or 0.001 kilopascal. The lyophilization may occur for a period of about 6, 12, 24, or 48 hours.

The super-cooled fluid of the method may comprise butane, pentane, hexane, heptane, octane, nonane, decane, or a mixture thereof. The super-cooled fluid may specifically be hexane. The super-cooled fluid may consist essentially of butane, pentane, hexane, heptane, octane, nonane, decane, or a mixture thereof. The super-cooled fluid may consist of butane, pentane, hexane, heptane, octane, nonane, decane, or a mixture thereof. The super-cooled fluid may be agitated to prevent freezing. In some aspects, the super-cooled fluid may be substituted with a super-cooled fluid having a low freezing point. The temperature of the super-cooled fluid may be below −40, −50, −55, −60, −65, or −70 degrees Celsius. The temperature may be specifically between −60 and −65 degrees Celsius. The super-cooled fluid may specifically have a temperature of less than −40 degrees Celsius.

The mixture of the method 100 may further comprise at least one cross-linking agent in addition to the regenerated silk fibroin and at least one medicament. The at least one cross-linking agent may be selected from the group consisting of ethylene glycol diglycidyl ether, poly(propyleneglycol)dyglycidyl ether, acetone, polyethylene glycol (PEG-400), or mixtures thereof. The method 100 may further comprise a step of mixing the cross-linking agent with an aqueous solution of the at least one medicament and the silk fibroin to form the mixture, wherein this step occurs prior to the injection of the mixture. This preliminary step may initiate gelation. The mixing may comprise vortexing the crosslinking agent and the aqueous solution. The aqueous solution may include a buffer, or specifically a basic buffer. The weight percent of silk fibroin in the mixture may be between 2 and 10 percent, between 3 and 6 percent, or between 4 and 5 percent.

The mixture may be injected in the method 100 using a needle positioned above the super-cooled fluid. The needle may be positioned at a sufficient distance above the super-cooled fluid to allow particle formation. The outlet of the needle may be positioned 1, 5, 10, 30, 40, or 50 millimeters above the super-cooled fluid. The mixture may be injected using a needle having an internal diameter between 0.2 and 0.8, between 0.3 and 0.6, or between 0.4 and 0.5 millimeters. The mixture may be injected at a pressure sufficient to produce a jet. For instance, the mixture may be injected between 80 and 300 kilopascals. The mixture may have a shear viscosity below at least 0.5, 1, 2, 3, 4, or 5 poise.

The medicament of the method 100 may be a therapeutic agent. For instance, the medicament may be selected from the group consisting of proteins, peptides, nucleic acids, nucleic acid analogs, nucleotides or oligonucleotides, peptide nucleic acids, aptamers, antibodies or fragments or portions thereof, antigens or epitopes, hormones, hormone antagonists, growth factors or recombinant growth factors and fragments and variants thereof, cell attachment mediators, cytokines, enzymes, antibiotics or antimicrobial compounds, viruses, toxins, prodrugs, chemotherapeutic agents, small molecules, drugs, and combinations thereof. The medicament may specifically be an antibiotic. If an antibiotic, the medicament may be selected from the group consisting of doxorubicin chloride, gentamicin sulfate, tobramycin sulfate, and kanamycin sulfate.

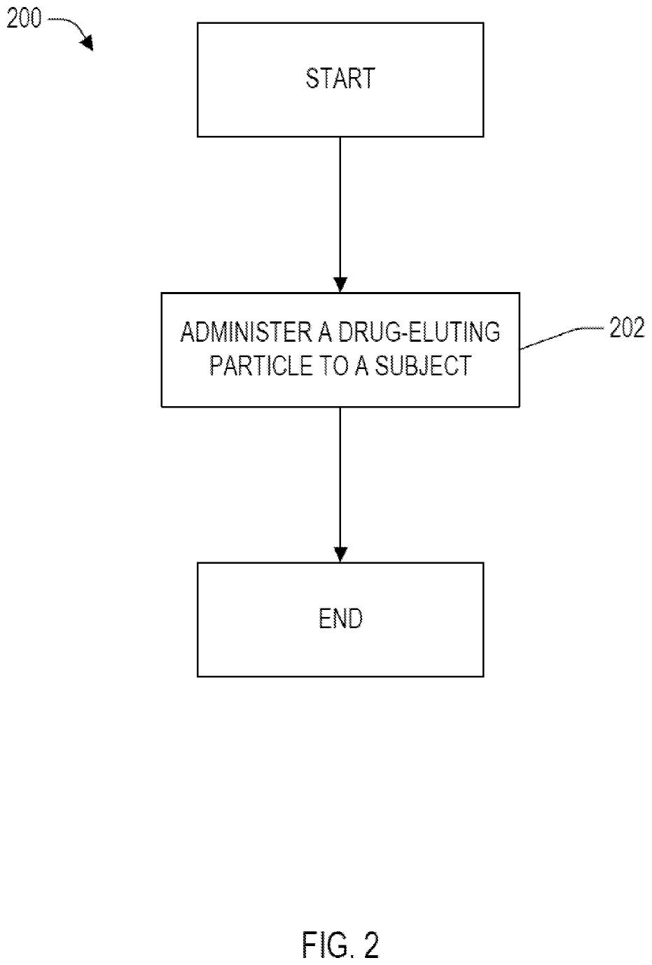

FIG. 2 depicts a method 200 of treating a subject. The method includes a first step 202 of administering a drug-eluting particles made by one of the methods described herein to the subject. The administered drug-eluting particles of the method may be in any product form consistent with the methods of formation described herein. For instance, the drug-eluting particles of the method may contain a medicament, such as an antibiotic. The drug-eluting particles may be administered via an injectable drug delivery system. The subject may be a mammal, such as a human subject. The administered drug-eluting particles may be biodegradable.

### Product Particles

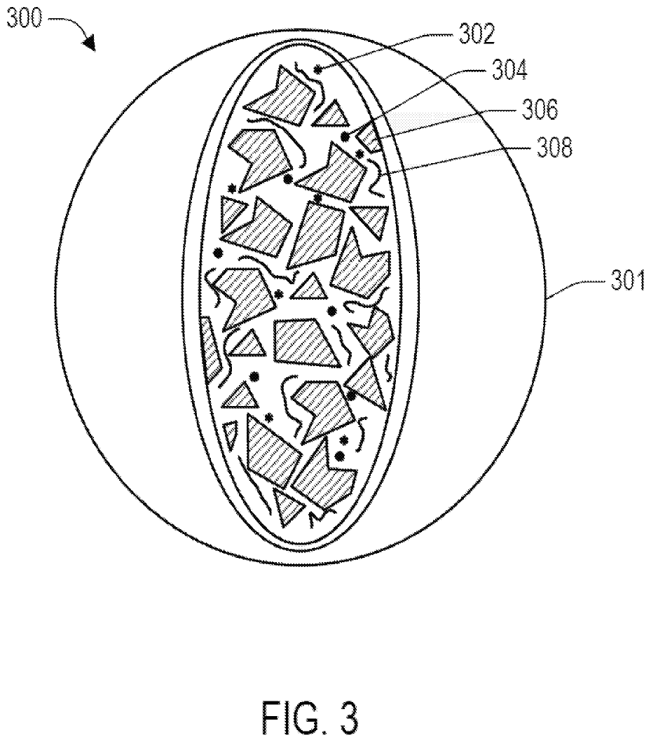

FIG. 3 depicts a representative schematic 300 of a regenerated silk fibroin particle 301 for drug elution. For illustrative purposes, the interior is shown using a cutout of the particle 301. The particle 301 has an interior composition comprising a cross-linking agent 302, a medicament 304, an ice template 306, and regenerated silk fibroin chains 308. The product regenerated silk fibroin particle 301 may be characterized by being formed by one of the methods provided herein. For instance, the particle 301 may be formed by the process comprising the steps of injecting a mixture into a super-cooled fluid to form drug eluting particles, the mixture comprising at least one medicament and regenerated silk fibroin. The average diameter of the particles may be between 300 and 800 μm. The average pore size of the particles may be between 0.1 to 10 μm. The particles may have a porous ice template. The particles may have pores of a sufficient size to release a medicament contained within the particle.

### Systems

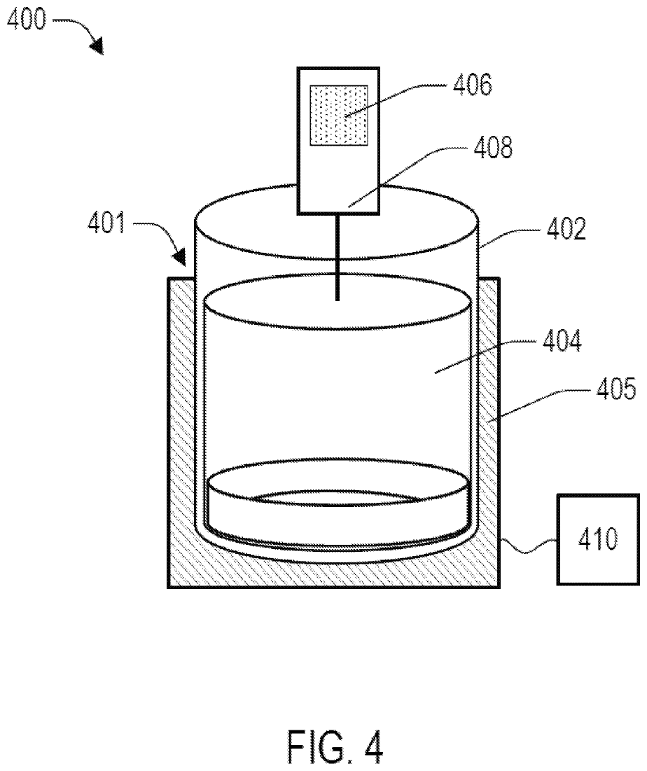

FIG. 4 depicts a system 400 for making drug-eluting particles. The system 400 includes a coagulation container system 401 configured to maintain a temperature below −40 degrees Celsius for a fluid retained therein; a super-cooled fluid 404 located within the coagulation container system 401; a source of a mixture 406 comprising at least one medicament and regenerated silk fibroin, the mixture optionally comprising a cross-linking agent; and an injection unit 408 positioned above a surface of the super-cooled fluid 404 and configured to inject the mixture 406 into the super-cooled fluid 404.

As shown in this depiction, the system 400 may further comprise a processor 410 coupled to the system and configured to control one or more of the following operational parameters: the temperature of the coagulation container system 401 and/or the super-cooled fluid 404; an injection pressure of the injection unit 408; an injection gage of the injection unit 408; a medicament concentration of the mixture 406; a regenerated silk fibroin concentration of the mixture 406; or a cross-linking agent concentration of the mixture 406.

The coagulation container system 401 may be configured to hold a fluid of variable volume and maintain a low temperature. For instance, the container system may maintain a temperature of about −40, −50, −55, −60, −65, or −70 degrees Celsius. The container system 401 may maintain such fluid temperatures using refrigeration techniques commonly known in the art, such as a dry ice bath or cooling jacket 405 surrounding a coagulation container 402. The super-cooled fluid located within the coagulation container system may have the same composition as the super-cooled fluids of the methods described herein. The coagulation container system may have a super-cooled fluid inlet and outlet, wherein super-cooled fluid may be introduced and removed from the system. This can allow for continuous production of particles without degeneration and contamination of the super-cooled fluid over time. The coagulation container system may have an agitation component, such as a stirrer, to maintain a liquid state super-cooled fluid.

The injection unit 408 may have a needle having an orifice or a comparable component for introducing the mixture. Such an orifice may be positioned about 1, 5, 10, 30, 40, or 50 millimeters above the top surface of the super-cooled fluid 404. The injection unit may be configured to continuously provide a uniform pressure to introduce the mixture. Suitable uniform pressures include those discussed for the methods herein. The coagulation system may further comprise an extraction system to assist in the removal of the particles. The extraction system may operate in a continuous, automated manner.

In another aspect, a system for making drug-eluting particles is provided. The system may comprise one or more components described herein for the automatic execution of any of the method steps described herein.

### EXAMPLES

The following examples set forth, in detail, ways in which the drug-eluting silk particles may be synthesized, and will enable one of skill in the art to more readily understand the principles thereof. The following examples are presented by way of illustration and are not meant to be limiting in any way.

### Example 1

A method for the preparation of large, microporous, drug-loaded particles was discovered and evaluated through an experimental study. High shear bollus injections of silk with cross-linker and drug colloids into super-cooled hexane were utilized to trigger phase separation of silk droplets, followed by immediate freezing at −60° C. A subsequent −20° C. freeze-thaw of the frozen droplets resulted in self-assembly (crystallization) of the silk. The silk particles developed an internal interconnected microporous morphology with 0.1-10 μm in diameter pores. The silk particles ranged in diameter from 100 to 1,300 μm, with particle mean diameter and polydispersity controlled by the starting concentration of the cross-linking agent and silk, the rheology of the reaction mixture, and the injection pressure (80-300 kPa). Cryogranulation provided a one-step process to produce microporous meso-scale silk particles with encapsulated drugs, such as doxorubicin chloride (DoxR), tobramycin sulfate (TS), kanamycin sulfate (KS) or gentamicin sulfate (GS). Almost 100% drug encapsulation efficiency was achieved in the process, and subsequent release profiles depended on the starting concentration of both the drug, silk, and pH of the elution medium. Kirby-Bauer tests and bioluminescent imaging confirmed the retention of anti-bacterial potency of the antibiotics pre-encapsulated in the cryo-particles, and macroparticles cytocompatibility towards human fibroblast and kidney cells.

### Materials

The following materials were used in the study: hexanes, for analysis (Thermo Fisher Scientific Co., US), ethylene glycol diglycidyl ether (EGDE, Tokyo Chemical Industry Co., Ltd.), poly(propyleneglycol)dyglycidyl ether (pPGDE, average Mn 500), gentamicin sulfate; acetone for HPLC, GC and residue analysis, ≥99.9%, tobramycin sulfate (all Sigma-Aldrich, 650501); kanamycin sulfate (Thermo Fisher Scientific Co., US); borate buffer was prepared using BupH™ Borate Buffer Packs (Thermo Fisher Scientific Co., US).

### Silk Fibroin Purification

To obtain regenerated silk fibroin (RSF), Bombyx mori cocoons were extracted for 45 min in boiling aqueous solutions of sodium carbonate (0.02 M), then rinsed quickly with distilled water three times to remove the excess salt and sericins. The extracted silk was loosened and dehydrated overnight at 25° C. under airflow. The dried silk mass was then dissolved in lithium bromide solution (9.3 M) for 4 h at 60° C. with occasional gentle agitation to yield a 20% w/v solution which was then cooled to 25° C. and dialyzed against distilled water for 48 h using cellulose dialysis tubing (molecular weight cutoff, MWCO, 3,500, Fisherbrand by Fisher Scientific Co., Pittsburgh, Pa.) to remove the salt. Thus obtained aq. solution of fibroin (ASF) was then subjected to the dialysis against borax buffer (pH 8.5) for 24 hours. The resulting basic solution of fibroin was checked visually for optical clarity and centrifuged and decanted 3 times at 9,000 rpm at 4° C. to remove residual solids or debris. The final concentration of aqueous silk solution was 9.5% wt., determined gravimetrically based on the residual dry solids weight.

### Cryogranulation

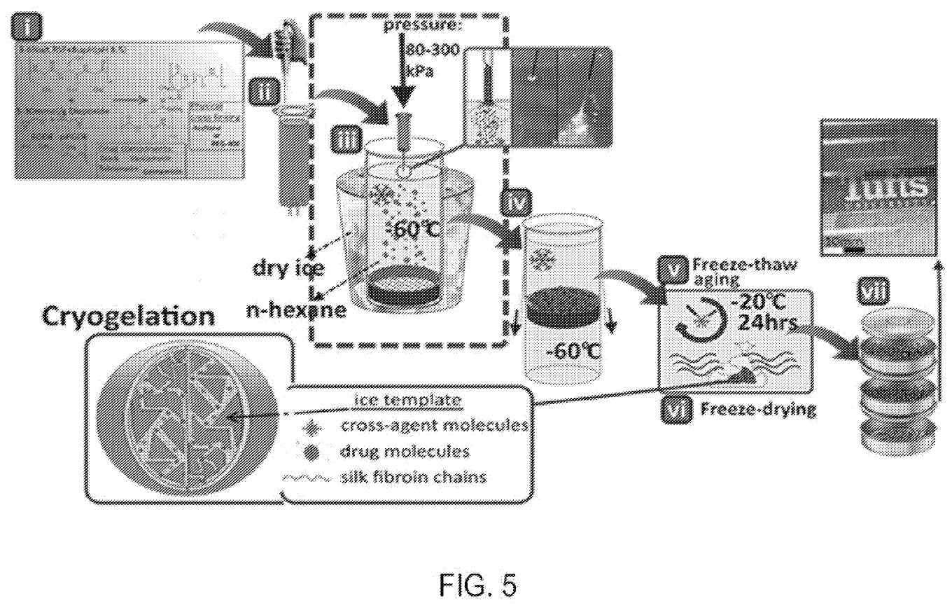

Each batch of RSFPs was produced using the custom designed silk cryogranulation assembly comprised of the coagulation bath, cooling jacket, 5 mL syringe, pressure switch unit, and the air compressor (FIG. 5). The coagulation bath was comprised of a glass cylinder (d×h: 90×230 mm) filled with 1200 mL of hexane and positioned coaxially inside a plastic beaker (d×h.: 150×180 mm) packed with crushed dry ice. The granule collection unit was comprised of a brass basket (d×h: 86×45 mm) draped with cellulosic liquid-permeable membrane (LensX™ 90 non-woven rayon blend tissues, Berkshire Corporation) deposited in the bottom section of the coagulation column. The setup was seated on top of an IKA 3810001 (Cole-Parmer Instrument Company, LLC) magnetic stirrer to maintain constant agitation of the freeze-hardening hexane bath at 100 rpm. The temperature of the pre-cooled coagulation bath was maintained at −60 to −65° C. The high shear injection unit was comprised of a 5-mL Cellink pressure tight syringe fitted with a 27 G needle; the inlet of the syringe was connected by polyethylene tubing (inner d=3 mm) to the pressure unit from the air compressor and a digital pressure controller built into the Inkredible 3D-Bioprinter system (Cellink Inc.). Gelation of silk was initiated by blending the desired amounts of ASF in BupH (pH 8.5) with either EGDE, pPGDE, acetone or PEG-400 by brief vortexing at 500 rpm for 10 seconds. Final concentrations of SF ranged 4-5.5% wt., concentrations of EGDE or pPGDE ranged 5-30 mmol of epoxy groups per 1 g of dry SF, concentrations of acetone and PEG-400 ranged 5-20% vol. The reactive suspensions were then transferred to the injection syringe. Syringe was then connected to the air compressor pre-tuned to the desired pressure from 80 to 300 kPa and the needle tip was fixed 10 mm above the surface of the hexane bath. The reactive systems were then immediately injected into the supercooled coagulation bath at a desired pressure, and the frozen silk droplets formed from the contact of the pressurized silk/cross-linker masterbatch with the supercooled hardening bath were collected at the bottom of the column. The frozen droplets were incubated in hexanes for 5 minutes then slowly withdrawn, quickly wrapped in the rayon cloth and incubated for 20 hours at −20° C. Traces of hexanes were stripped off from the RSFPs by air convection in the cooling chamber and later by lyophilization for 24 hours at 0.06 mbar. The RSFPs were then washed with 200-x excess of DI water to remove any unreacted protein and the cross-linkers. The cryo-RSFPs obtained could further be fractioned by particle size via dry sieving using nested sieve columns, each sieving plate 80 mm in diameter, with the following screen openings (in μm): 1,400; 425; 355; 212; 180.0; 150.

### Encapsulation of DoxR and Antibiotics in RSFPS

Doxorubicin chloride (DoxR), gentamicin sulfate (GS), tobramycin sulfate (TS), or kanamycin sulfate (KS) were dissolved in BupH buffer (pH 8.5) and blended with the stock ASF and 10% vol. acetone to obtain final drug concentrations in range from 1 to 20 mg/mL. The resulting drug/RSF/cross-linker was immediately injected at 150 kPa into the supercooled hexane bath. The frozen RSF/drug droplets were harvested from hexane, incubated for 20 hours at −20° C. and freeze-dried to remove traces of hexane, water, and acetone. The total antibiotic content in the RSF beads was determined gravimetrically by subtracting the average dry weight of the blank RSFPs from the average dry weight of drug-loaded RSFPs.

### Preparation of RSFPS Modified with NRSFS

To synthesize silk nanoparticles, 45-minute extracted RSF solution in BuPh buffer (pH 8.5) was used. In brief, the ASF was diluted to 5% wt. of RSF and mixed dropwise (0.2 drop/s; ca. 100 μL/drop) to pure acetone, with the final acetone fraction accounting for 80% (v/v). Silk particulated were left to precipitate overnight, then centrifuged for 30 min at 9,000 rpm, followed by aspiration of the supernatant and re-suspension/vortexing of the pellet in 35 mL of DI water. Crude SF suspensions were then sonicated for 30 s at 30% amplitude with a Branson Digital Sonifier 450 (Branson Ultrasonics, Danbury, Conn., USA). The centrifugation-washing-suspension cycle was repeated twice to obtain silk nano-suspensions (nRSF). A 0.5-mL aliquot of the n-RSF was freeze-dried and the dry solids content was determined gravimetrically. Prior to cryo-granulation, the n-RSF suspension was subjected to a 15 s sonication at 30% amplitude followed by size and zeta potential assessments with a Microbrook 2000 L laser particle-size analyzer (BrookHaven Instruments Corporation, NY). A calculated volume of the n-RSF suspension was mixed with the DoxR for 2 hours at 25° C. with constant agitation (200 rpm). The resulting system, containing free DoxR and DoxR-loaded nRSFs was added to the ASF stock to reach concentrations of n-RSF in the system in range from 3 to 6 mg/mL. These mixtures were immediately mixed with 10% vol. acetone and subjected to the above cryo-granulation process at 150 kPa, to produce nano-modified DoxR-loaded RSFPs.

### Fourier Transform Infrared Spectroscopy

IR-Spectra of the lyophilized RSFPs as well as pure RSF and cross-linking agents (in liquid from) were acquired using a single bounce diamond attenuated total refractance (ATR) module on a Fourier-transform infrared (FTIR) spectrometer (JASCO FTIR 6200 Spectrometer, JASCO, Tokyo, Japan) equipped with a MIRacle Ge crystal cell in the reflection mode. Air was used as the reference and subtracted automatically from sample readings. Each sample was analyzed in the frequency range from 400 to 4,000 cm⁻¹ with each measurement adding 32 interferograms with a resolution of 4 cm⁻¹. The fractions of secondary structures, including random coil, alpha-helices, beta-pleated sheets, and turns, were ascribed to the amide I region from 1,595 to 1,705 cm⁻¹. Random coil, alpha-helices and β-turns were ascribed to the absorption bands in the frequency ranges of 1,638-1,655 cm⁻¹, 1,656-1,663 cm⁻¹ and 1,663-1,695 cm⁻¹, respectively.

### Differential Scanning Calorimetry

An estimate of 3 mg of lyophilized RSFPs was packed in aluminum pans and measured in a Q600 TGA/DSC instrument (TA Company, New Castle, Del.). The chamber of the instrument was purged with dry nitrogen at 50 ml/min. The instrument was calibrated with indium for heat flow and temperature. Aluminum and sapphire reference standards were used for calibration of the heat capacity. The measurements were performed using a standard mode at a heating rate of 8° C./min.

### Rheology

The shear viscosity of each freshly prepared injectable RSF/cross-linker system was measured by rotational rheometry (AR 2000, TA Instruments) using two 50 mm circular titanium plates at 25° C. The normal force applied on the sample during lowering of the top plate was limited to 0.1 N. The shear rate was linearly increased from 0.1 to 5,000 l/s at the steady state deformation of 15%.

### Size Distributions of RSFPS

Direct estimation of mean size distribution of RSFPs was performed using a combination of fluorescence microscopy and the following automated image processing with the ImageJ. The micrographs of cryo-RSFPs were recorded using a BZ-X710, All-in-One Fluorescence Microscope (Keyence, USA) with a DAPI filter and at 42× magnification. The recorded scans were further stitched and processed by contrasting and grain analysis using the ImageJ open access software.

### Loading Efficiency and Release of DoxR

A 100 mg load of DoxR/RSF particles with sizes between 200-800 μm was sealed in water-permeable rayon membranes and incubated in 5 mL of PBS buffer at 37° C. and constant agitation of the elution medium at 50 rpm. At determined time points, 100 μl aliquots of the elution medium were collected and the absorbance at 477 nm (DoxR) and 480 nm (DNR) was measured. The concentration of DoxR at each time point (20 min. to 14 days) was determined by extrapolation of the absorption to standard curves obtained for known concentrations of drug from 0.01 to 1 mg/mL. Non-drug cryo-RSFPs were used as negative controls.

### Residual Antibacterial Activity

Antimicrobial activity of tobramycin sulfate (TS), gentamicin sulfate (GS) and kanamycin sulfate (KS) encapsulated in RSFPs sized 100-500 mcm was assessed by Kirby-Bauer disc diffusion tests, performed according to methods outlined in the NCCLS Approved Standard M2-A7. A stock suspension of bioluminescent S. aureus Xen29 was prepared in sterile physiological solution and turbidity (expressed as optical density; OD) was adjusted against the McFahrland standard to match the 0.5 level. A sterilized cotton swab was immersed in the bacterial suspension, and a lawn of bacteria was applied on the Luria Bertani (LB) plates. A 10-mm biopsy punch was used to cut two circular fragments from each of the freshly infected plates, and both fragments were coated with antibiotic-loaded RSFPs by quick blotting against the pool of particles. The freshly mounted samples were inserted back into the circular slots of the bacteria-seeded plates and further cultured for 48 hours at 37° C. In vitro imaging of the plates was performed after 24 hours of incubation using a Caliper IVIS Lumina II system (Caliper life Science, America). The exposure time (1 s), excitation filter (430 nm), and emission filter with emission wavelength from 575 to 650 nm (DsRed) were set prior to detection.

### Scanning Electron Microscopy (SEM)

The morphology of the particle's surface and inner phase of was visualized by field emission scanning electron microscopy (FE-SEM, JEOL, JSM-6490, Japan) at a voltage of 5 kV. Before analysis, the freshly lyophilized particles were positioned onto a carbon paint adherent to the aluminum sample holder. Cryo-sectioning of large (500-1,000 μm) meso-particles was used to prepare samples for the in-depth study of microporous morphology: RSF-particles were quenched in liquid nitrogen and manually crushed using a pre-cooled porcelain mortar and pestle. The resulting crushed samples were freeze-dried and fixed on the sample holder. All samples were made conductive by sputtering a thin layer of gold onto their surface.

### Preparation of the RSF-Scaffolds

The silk-based scaffolds were fabricated using the cryogelation protocol to closely mimic the conditions applied to RSF during the cryo-granulation procedures. Solutions of RSF were mixed with either EGDE or acetone, to give a final 4.5% wt. of RSF, 30 mmol/g of EGDE or 20% vol of acetone. These mixtures were molded in a 48-well plate and frozen using dry ice as coolant. The vitrified samples were incubated at −20° C. overnight in hexane, then thawed at 4° C. and washed extensively with DI water to obtain the chemically (EGDE) or physically (acetone) cross-linked RSF-sponges, sized 8 mm in diameter and 5 mm thick. Prior to cell seeding, the compressive modulus of each type of scaffold was measured at 10 Hz with 25% compressive strain using the TA Instruments RSA3.

### Cell Culture

Immortalized human renal cortical epithelial cells (RPTEC/TERT1, ATCC, Manassas, Va., USA) and human fibroblasts (HNF) were cultured in DMEM:F12 (ATCC), 5 pM triiodo-L-tyronine sodium salt (Sigma-Aldrich, St. Louis, Mo., USA), 10 ng/ml recombinant human epidermal growth factor (Life Technologies, Grand Island, N.Y.), 1% ITS (Life Technologies), 25 ng/ml prostaglandin El (Millipore, Billerica, Ma.), 25 ng/ml hydrocortisone (Sigma-Aldrich), 0.1 mg/ml G418 (Life Technologies), and penicillin-streptomycin (Life Technologies). The RSF sponges were sterilized by autoclaving and then seeded with 2.5×10⁵ cells/scaffold (P=4). The cultures were maintained for 21 days with the RPTEC media.

### Cell Viability

Cell viability was assessed on Days 10 and 21 using a LIVE/DEAD™ Viability/Cytotoxicity Kit for mammalian cells (L3224, Thermo Fischer Scientific). PBS (300 μl) supplemented with 10 mM calcein AM green and 1 mM ethidium homodimer-1 was added to the cells and incubated for 45 min. The samples were visualized using a BZ-X710, All-in-One Fluorescence Microscope (Keyence, USA).

### Immunofluorescence Studies

After 10 and 21 days of culture, the RSF-constructs were fixed with 10% formaldehyde for 15 min, washed with three times with PBS. For immunohistochemistry, the constructs were blocked with 10% bovine serum albumin for 30 min at room temperature and incubated with primary antibodies against E-cadherin (ab1416/abcam), actin (ab3280/abcam), smooth muscle actin (ab5694/abcam) and 4′,6′-diamidino-2-phenylindole (DAPI/D1306/Thermo Scientific) was used to stain the nucleus. Alexa Fluor 546 goat anti-mouse IgG (1:200, Invitrogen) was used for fluorescent labelling. Following this step, specimens were rinsed three times with PBS/Triton and mounted for visualization by Confocal Laser Scanning Microscope (Leica TCS SP5, Germany).

### Statistics

Particle size measurements were obtained in triplicate for two parallel batches of RSFPs, fabricated with the given set of parameters, with and expressed as mean±standard deviation (SD). Rheological properties of the silk/cross-linker colloids and the drug release kinetics were acquired as three parallel measurements.

### Results and Discussion

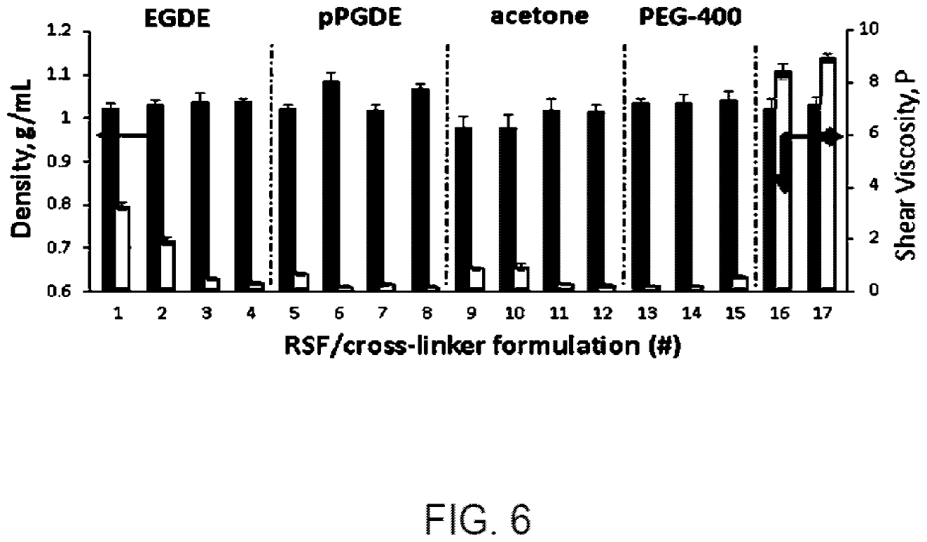

The main steps of the experimental batch-based silk cryo-granulation process are presented in FIG. 5. The mixture of precursors was blended, consisting of RSF, a cross-linking agent chosen from EGDE, pPGDE, acetone or PEG-400, and one of the target therapeutic compounds chosen from doxorubicin chloride (DoxR), gentamicin sulfate (GS), kanamycin sulfate (KS) or tobramycin sulfate (TS). The mixture was then injected through a 27 G-needle at pressures of 50-300 kPa into the hexane bath refrigerated at −60 to −65° C. One of the unique aspects of the current granulation technique relies on the instantaneous splintering of the reactive RSF jet into discrete silk droplets upon contact with the water immiscible organic phase. Without being bound by theory, this approach contrasts with the microfluidic processes where droplet separation is via a laminar take-off of the liquefied RSF within the co-flow granulation devices. Prior continuous microfluidic methods exploit the Rayleigh-Plateau instability within the stream of RSF to trigger droplet formation. Here the size and morphology of the resulting particles may be controlled by the viscosity of the contact liquids, and the discrete droplets are achieved by RSF solutions with viscosities above 10 Poise. Bollus injections of the plain 3-6% wt. RSF solutions produced continuous fibers 400-1,000 μm thick. Dynamic rheometry showed that the pure RSF solutions had shear viscosities in range 8-9 P, several times higher than 0.2-3.5 P for the RSF/cross-linker mixtures (FIG. 6). Formation of discrete droplets was enabled in the reactive mixtures of RSF with either 10-20% vol. of acetone, 5-30 mmol/g of EGDE or PPGDE, or 5-15% vol. PEG-400, reduced shear viscosity of 0.2-4 P and densities of 1.0-1.15 g/cm³ (FIG. 6). This observation highlights the difference between cryo-granulation and microfluidic techniques in terms of rheological properties.

Hexane has a melting point between −94 and −96° C. and remains liquid upon cooling with dry ice to −60° C. and −65° C., enabling rapid separation of the RSF 50-2,000 μm droplets and stabilization of external shape and dimensions through freezing. Unlike previous cooled petroleum ether bath methods implemented for the continuous production of PVA-based meso-spheres, the process described in the present work supports processing of up to 10 mL/min of the starting RSF mixture, significantly faster compared to the prior co-flow assisted jetting devices. The residual hexane was successfully removed from the frozen RSFPs by air convection in the freezing chamber, followed by 24-hour lyophilization.

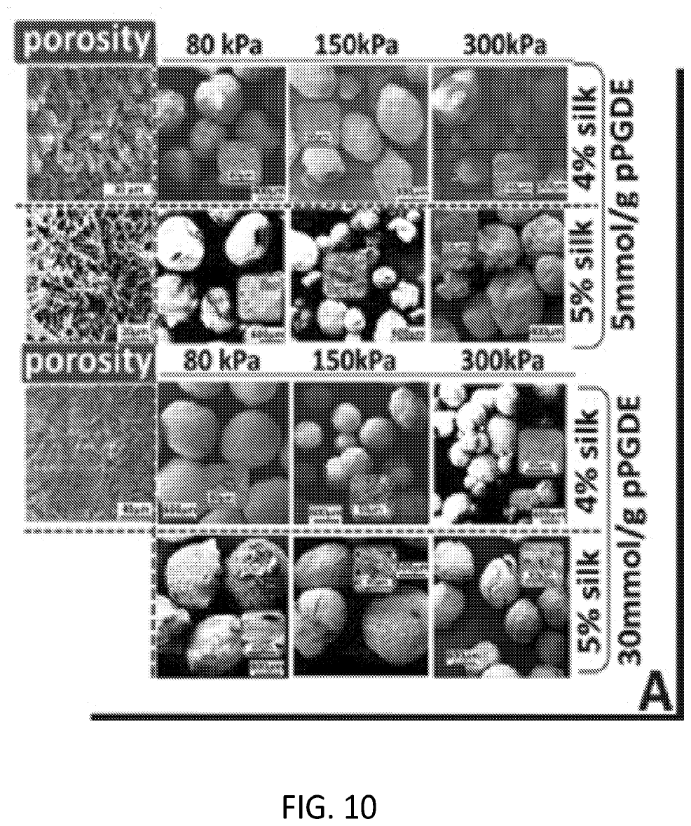

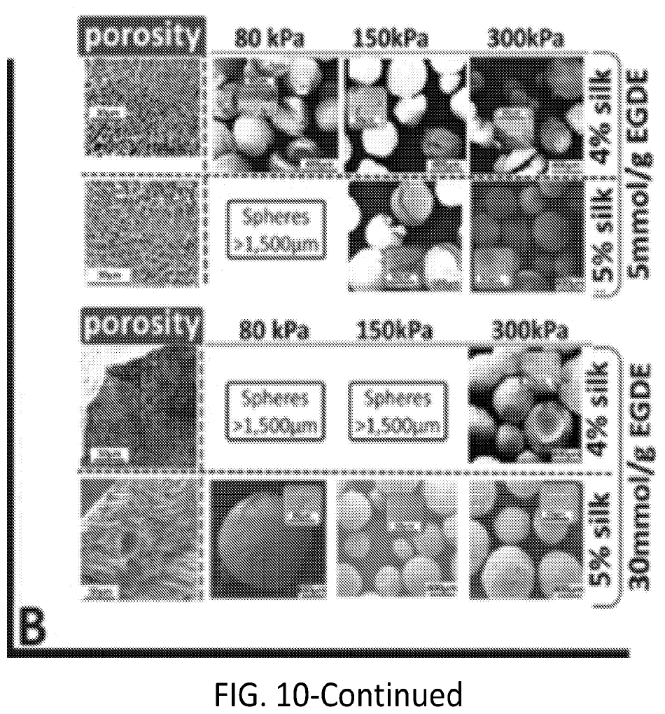

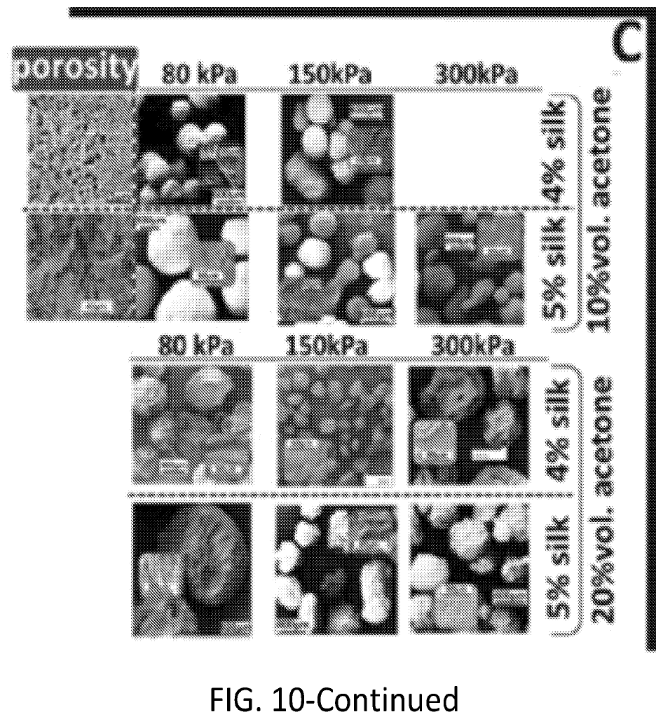

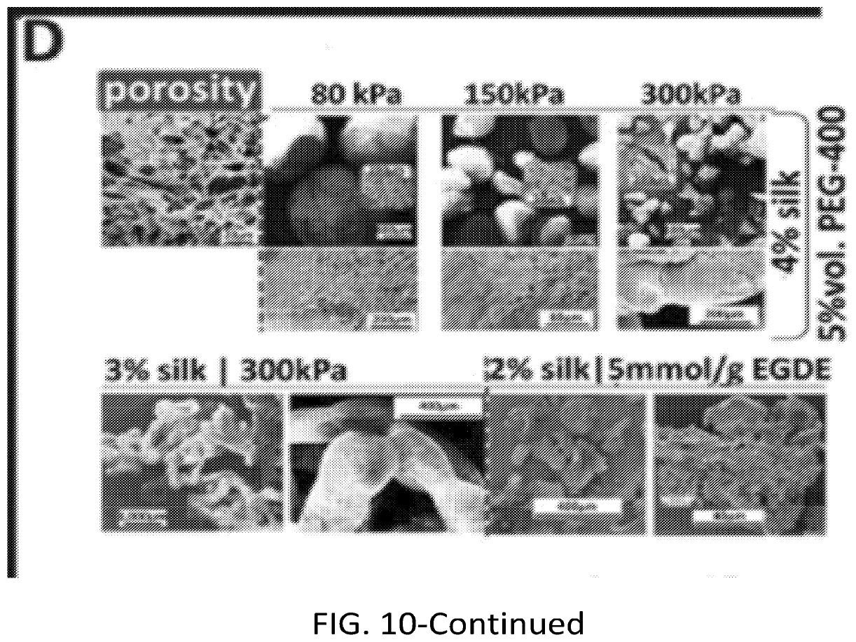

Concentrations of RSF above 6% wt. resulted in premature gelation and clogging of the injector's needle, and below 2.5% wt. RSF poorly shaped granules were produced with a tendency to shrink and disintegrate upon drying (FIG. 10D). The rheological behavior can be controlled to some extent based on molecular weight of the silk. Thus, we used reactive mixtures with 30 mbs (higher molecular weight than the 60 mbs) RSF with shear viscosities between 10-15 P, which produced continuous fibers, similar to those obtained from the 6% wt 45 mbs RSF. Injections below 150 kPa resulted in dripping of the reaction mixture, yielding large silk droplets which did not demonstrate further break up upon contact with the hexane bath and quickly froze, yielding spherical beads of around 2,000 μm. In this work we assessed the morphology and physico-chemical properties of the particulate materials produced only in course of the jetting regime. Dripping-mode injections were observed in case of the 45 mbs RSF-containing systems despite viscosities as low as 4-0.1 P, mixtures of 30 mmol/g of EGDE or PPGDE or 15% vol of PEG-400 to RSF (especially at concentrations above 5% w), and lead to rapid precipitation of the protein which tended to clog the needle, see Table 1. Similar issues were found with the acetone/RSF mixtures, which typically remained cryo-injectable at pressures above 100 kPa, while dripping at pressures below 80 kPa.

TABLE 1A-D
Statistical data for silk particle size distributions determined by
automated microscopy analysis via a combination of fluorescent imaging and ImageJ calculations.
A. Cross-linking with EGDE

| [RSF], % wt. | [EGDE], Mmol/g | P, Pa | D10, μm | D90, μm | Mean D, μm |
|---|---|---|---|---|---|
| 4.0 | 5 | 80 | 358 ± 41 | 823 ± 121 | 651 ± 39 |
| | | 150 | 362 ± 15 | 960 ± 65 | 684 ± 43 |
| | | 300 | 305 ± 1.9 | 688 ± 27 | 492 ± 54 |
| | 30 | 80 | Dropwise injection | | |
| | | 150 | Dropwise injection | | |
| | | 300 | 189 ± 51 | 686 ± 27 | 359 ± 27 |
| 5.5 | 5 | 80 | Dropwise injection | | |
| | | 150 | 371 ± 41 | 762 ± 43 | 512 ± 68 |
| | | 300 | 277 ± 68.6 | 805 ± 83 | 566 ± 42 |
| | 30 | 80 | Dropwise injection | | |
| | | 150 | 551 ± 34 | 1091 ± 89 | 786 ± 65 |
| | | 300 | 222 ± 23.7 | 790 ± 120 | 510 ± 39 |

B. Cross-linking with pPGDE

| [RSF], % wt. | [pPGDE], mmol/g | P, Pa | D10, μm | D90, μm | Mean D, μm |
|---|---|---|---|---|---|
| 4.0 | 5 | 80 | 382 ± 48 | 936 ± 74 | 661 ± 236 |
| | | 150 | 275 ± 11 | 774 ± 17 | 526 ± 225 |
| | | 300 | 201 ± 14 | 554 ± 42 | 366 ± 132 |
| | 30 | 80 | Dropwise injection | | |
| | | 150 | Dropwise injection | | |
| | | 300 | 326 ± 25 | 650 ± 32 | 537 ± 136 |
| 5.5 | 5 | 80 | Dropwise injection | | |
| | | 150 | 385 ± 28 | 942 ± 113 | 655 ± 240 |
| | | 300 | 405 ± 29 | 1000 ± 40 | 699 ± 244 |

| | | |
|---|---|---|
| 30 | 80 | Dropwise injection |
| | 150 | Dropwise injection |
| | 300 | Dropwise injection |

C. Cross-linking with acetone

| [RSF], % wt. | [acetone], % vol. | P, Pa | D10 | D90 | Mean D, x |
|---|---|---|---|---|---|
| 4.0 | 10 | 80 | 413 ± 15 | 774 ± 133 | 625 ± 129 |
| | | 150 | 322 ± 47 | 665 ± 7 | 510 ± 51 |
| | | 300 | 265 ± 28 | 581 ± 24 | 429 ± 63 |
| | 20 | 80 | Dropwise injection | | |
| | | 150 | 408 ± 5.3 | 584 ± 16 | 508 ± 17 |
| | | 300 | 359 ± 66 | 630 ± 24 | 501 ± 21 |
| 5.5 | 10 | 80 | Dropwise injection | | |
| | | 150 | Dropwise injection | | |
| | | 300 | 500 ± 5 | 930 ± 125 | 730 ± 180 |
| | 20 | 80 | Dropwise injection | | |
| | | 150 | 425 ± 12 | 872 ± 47 | 653 ± 177 |
| | | 300 | 400 ± 71 | 915 ± 17 | 627 ± 192 |

D. Cross-linking with PEG-400

| [RSF], % wt. | [PEG400], % vol. | P, Pa | D10 | D90 | Mean D, |
|---|---|---|---|---|---|
| 4.0 | 5 | 80 | Dropwise injection | | |
| | | 150 | 534 ± 46 | 986 ± 114 | 758 ± 222 |
| | | 300 | 451 ± 42 | 849 ± 67 | 594 ± 139 |
| | 15 | 80 | Dropwise injection | | |
| | | 150 | 670 ± 22 | 1,211 ± 55 | 921 ± 265 |
| | | 300 | 421 ± 55 | 827 ± 1 | 636 ± 167 |
| 5.5 | 5 | 80 | Dropwise injection | | |
| | | 150 | 499 ± 98 | 1074 ± 17 | 776 ± 227 |
| | | 300 | 514 ± 26 | 931 ± | 701 ± 47 |

| | | |
|---|---|---|
| 15 | 80 | Premature gelation, needle clogging |
| | 150 | Premature gelation, needle clogging |
| | 300 | Premature gelation, needle clogging |

Blank spaces designate conditions that resulted in macro-spheres with diameters exceeding 1,500 μm.

Apart from the emulsification of the starting RSF solutions, the additions of either diepoxides (EGDE, pPGDE) or desolvating agents (acetone, PEG-400) facilitated further solidification of silk within the frozen droplets. Structural stabilization of silk was achieved via internal cryogelation by a 20-hour freeze-thaw aging of the quenched droplets at −20° C. Cryogelation of RSF was triggered by covalent cross-linking with 5 or 30 mmol/g of EGDE. Identical concentrations of the oligomeric pPGDE were used to differentiate between cross-linker type and the morphology of the resulting epoxide-cross-linked RSFPs. Physical cryogelation of RSF within the frozen droplets was achieved through addition of acetone or PEG-400, which initiate the transition of the water-soluble Silk I conformation (characterized by a mixture of random coil, α-helix and β-turn structures), into stable and water insoluble Silk II, predominantly composed of the β-sheets. The lyophilized RSFPs, cross-linked both physically and chemically, were water-insoluble and had a powdery consistency. According to the post-granulation gravimetry, internal cryogelation with acetone resulted in gel fraction yields (mass ratio between the starting dry weight of the RSF being cross-linked and the final RSFPs weight) in range from 85 to 95%, whereas cryogelation in the presence of PEG-400 cross-linked between 68 and 75% RSF, while the EGDE- and pPGDE-assisted cryogelation cross-linked between 45 and 51% and between 59 and 74% of the starting silk, respectively. Thus, acetone/RSF mixtures were used as the matrix media to pre-disperse drug compounds to produce drug eluting beads.

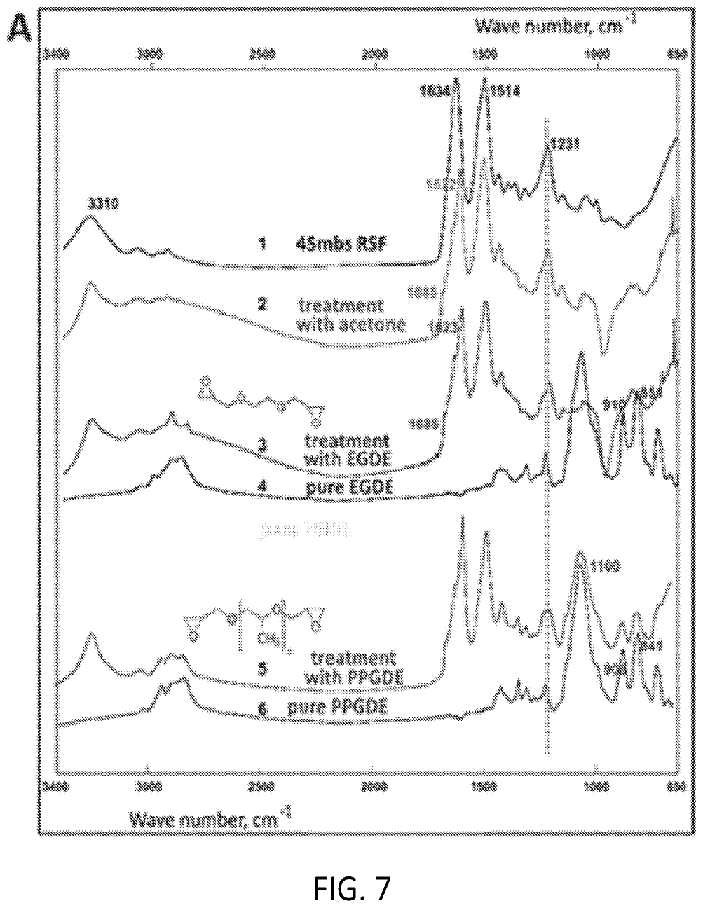

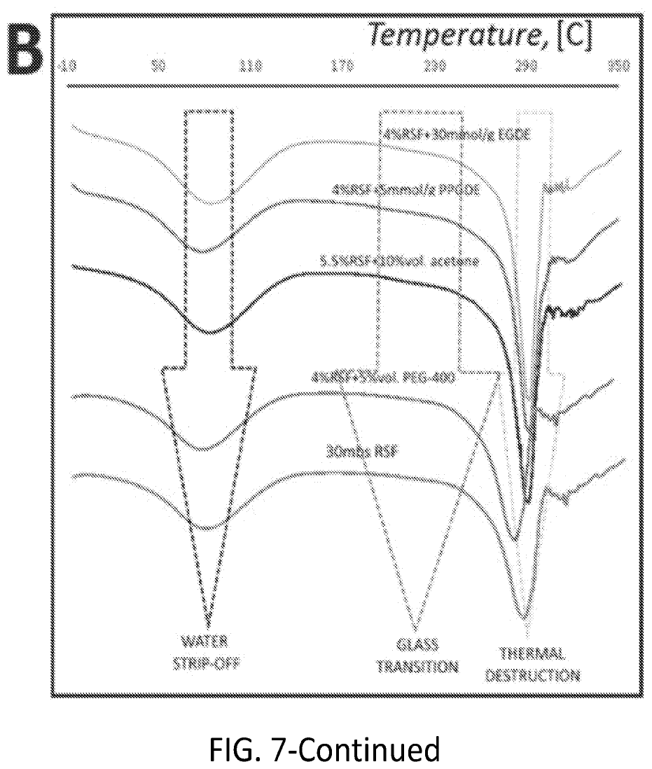

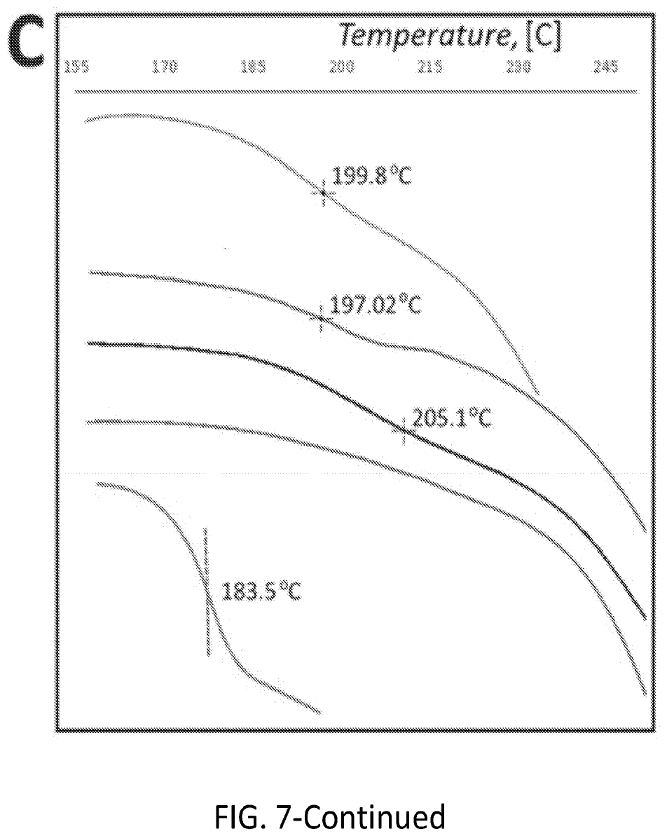

The FTIR-spectra of lyophilized water soluble RSF (FIG. 7, curve 1) showed characteristic vibration bands between 1,630 and 1,650 cm−1 for amide I (C—O stretching) indicating the presence of primarily random coil and/or α-helix conformations, 1,540-1,520 cm−1 for amide II (secondary N—H bending) and 1270-1230 cm⁻¹ for amide III (C—N and N—H functionalities). The RSFPs cross-linked with acetone were similar to that of the lyophilized RSF and contained chemical shifts of the above absorption frequencies as shoulders to the peaks at 1,685 and 1,622 cm⁻¹, 1,580.0 and 1,238.4 cm⁻¹, respectively, reflecting the formation of β-sheet structures (FIG. 7, curve 2). The FTIR-spectra of the EGDE- and PPGDE-stabilized RSFPs (FIG. 7, curves 3 and 5, respectively) with reference to the spectra of the pure cross-linkers (FIG. 7, curves 4 and 6, respectively) displayed main peaks at 1,623 cm⁻¹ assigned to a β-sheet conformation, as well as shoulders at 1,660 and 1685 cm−1, which could be assigned to α-helix and β-turn conformations, respectively. This indicated a conformational transition from random coil (Silk I) to a β-sheet (Silk II) structure in the frozen droplets of RSFP precursors. Without being bound by theory, the build-up in β-sheet content in silk cryogels is suspected of arising from the cryo-concentration effect causing a nearly 10-fold increase of RSF concentration within the unfrozen liquid microphase. This process was accompanied by the loss of molecular mobility of silk macromolecules which may be due to both the physical entanglements and covalent etheric cross-links between the protein chains. Diepoxide cross-linking of RSF can be traced by new bands at 1,040-1,100 cm−1 upon gelation, which were assigned to the ether stretching bands of EGDE and PPGDE (FIG. 7, curves 3&4, respectively) intermolecular cross-links.

Differential scanning calorimetry was used to characterize the thermal properties of the RSFPs. All particles released water, as indicated by a broad endothermal peak around 60-80° C. Further, heating the silk samples to 350° C. resulted in thermal degradation of the cross-linked protein matrix as indicated by the sharp exotherms at 285-295° C. (FIG. 7, b). Cryogelation of RSF caused an increase of the RSFP glass transition temperature by ca. 15-20° C., compared to the non-treated silk (FIG. 7, c). This result indicated that the intermolecular interactions between the amorphous domains of RSF were restricted by the cross-links induced by exposure to either diepoxides or acetone. Cross-linking of the RSFPs with acetone yielded the highest Tg values, up to 205° C., compared to the glass transition at ca. 198-200° C. for the pPGDE, PEG-400EGDE-cured RSFPs, which could indicate β-sheet rich crystalline fractions of silk fibroin in these samples.

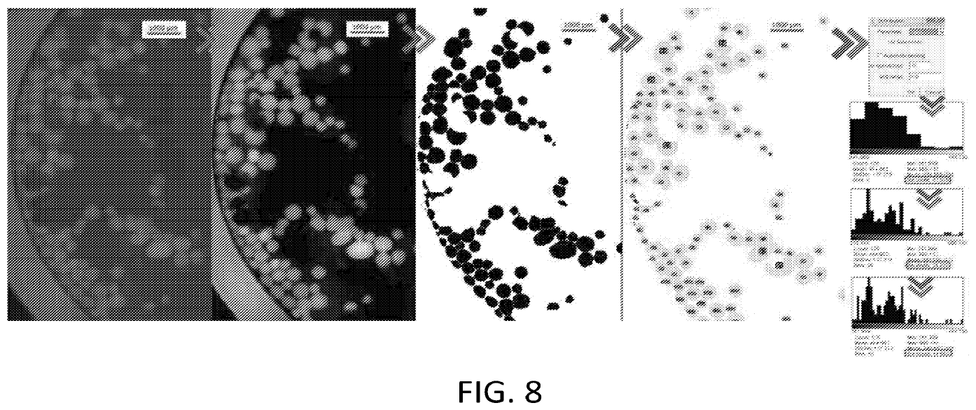

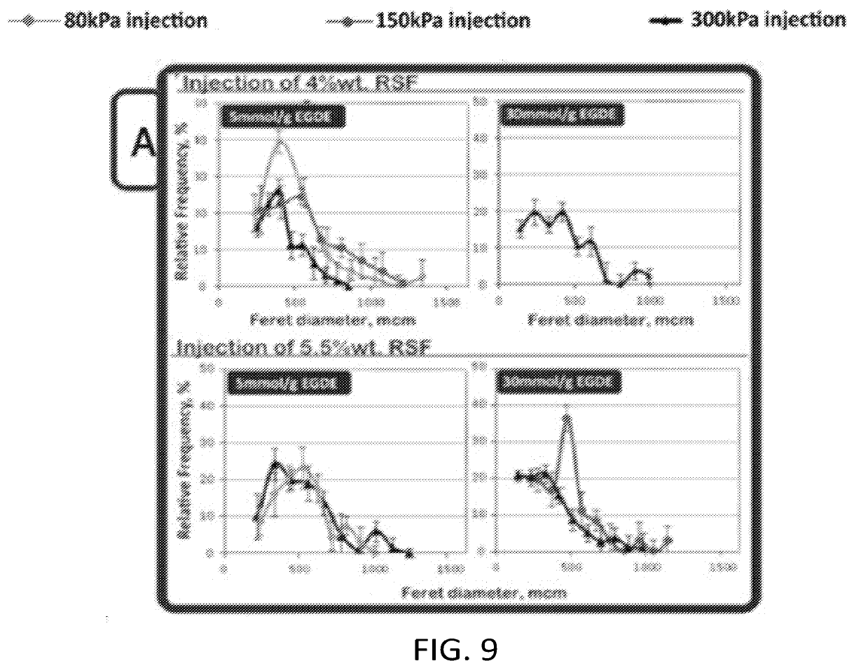

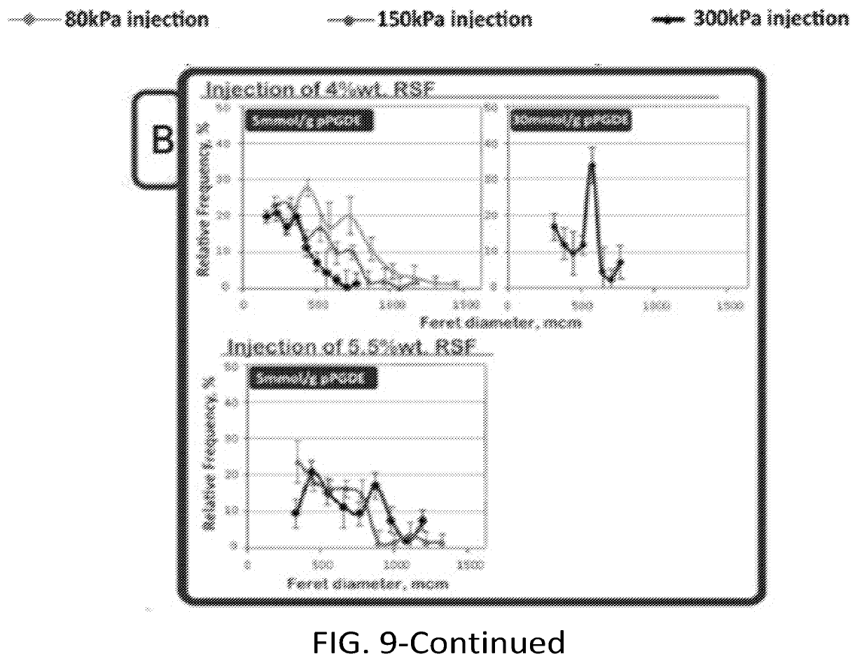

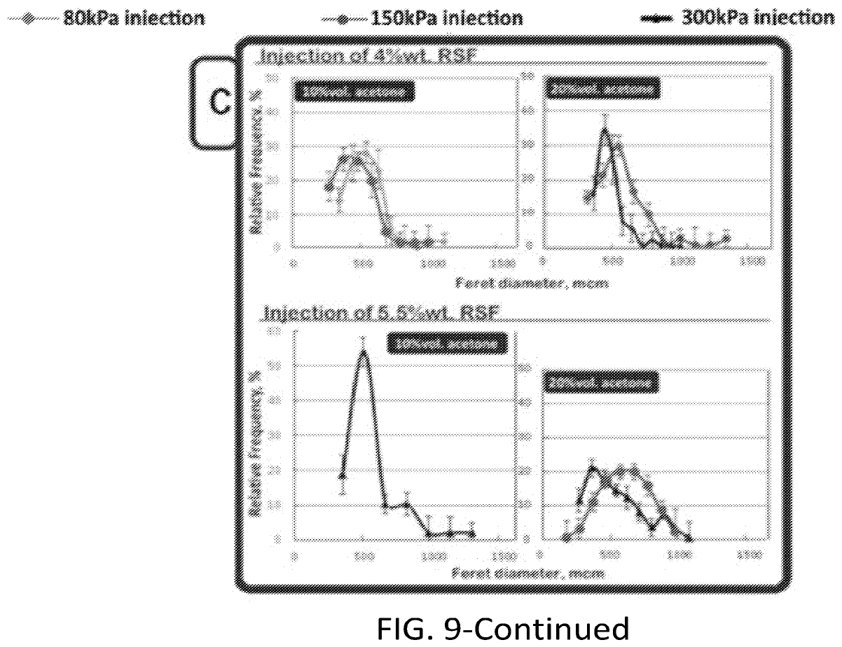

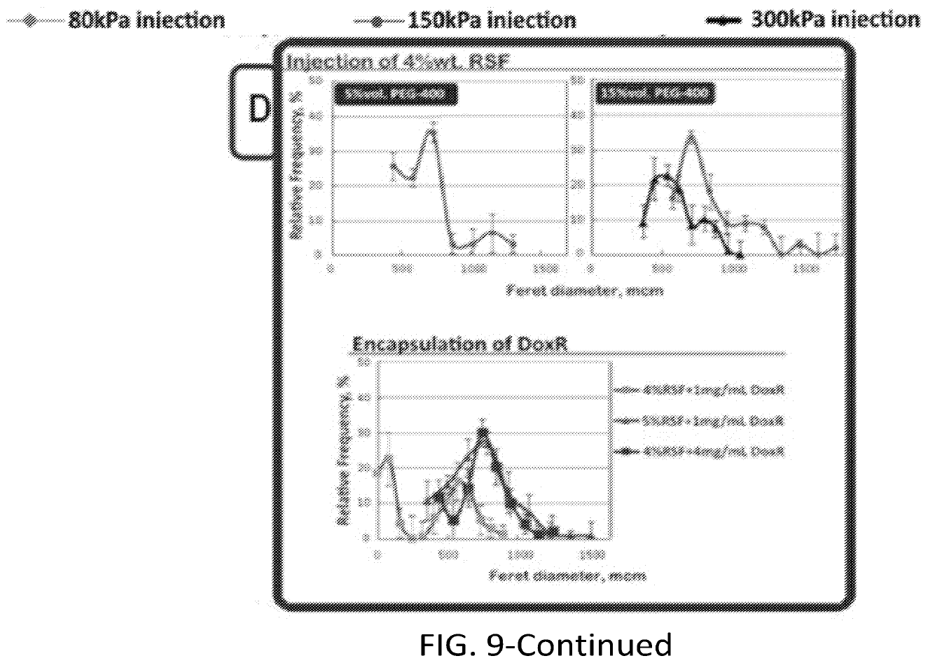

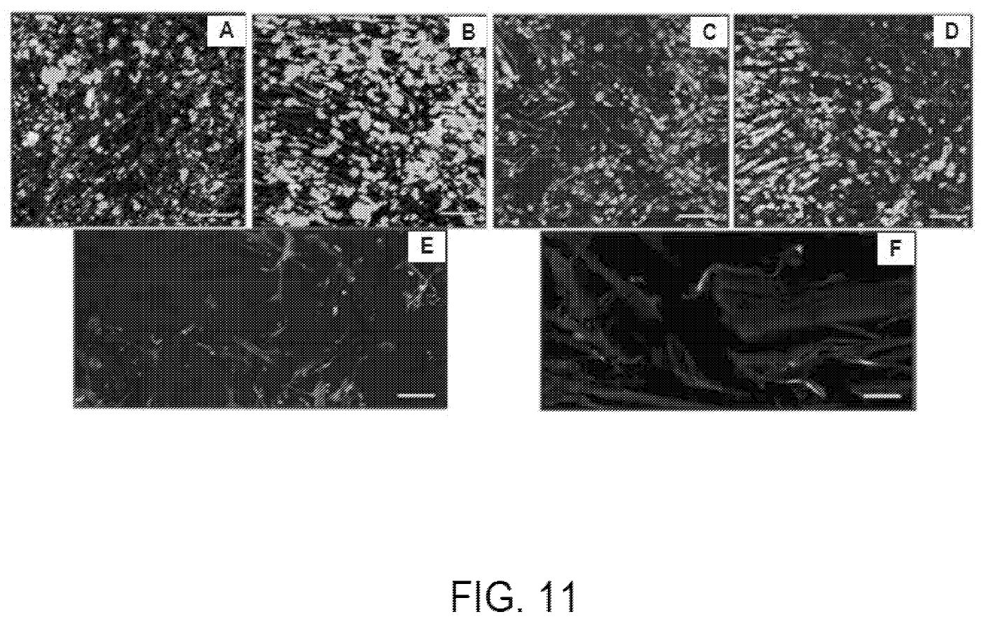

Without being bound by theory, the dynamics of droplet break up, as well as droplet size and shape, are governed by the competition between the interfacial tension holding the jet of the silk colloid intact, and the Rayleigh-Plateau instability within the focused jet, which increases at higher flow rates. The cryo-granulation used here controls the size and polydispersity of the final RSFPs by both flow rate of the reactive colloid (injected under the pressures tuned within the range of 80 to 300 kPa) and the rheological properties of the colloids, in turn, defined by the cross-linker type and the cross-linker/RSF mass ratio. FIG. 11 shows representative RSFP fluorescent micrographs converted into binary offsets and analyzed for mean particle size, expressed as the Feret diameters. The influence of the fabrication parameters on the resulting size distribution of RSFPs was fingerprinted by the relative frequency profiles (FIGS. 8, 9). The size distributions of water-swollen RSFPs could be varied over a wide range, between nominal D10/D90 cutoffs of ca. 200 and 1,000 μm, depending on the injection pressure and the composition of the injectable system, see Table 2.

TABLE 2
Formulations subjected to cryogranulation numbered with respect to
the type and the starting concentrations of the cross-linker and RSF.

| | | Type | | | |
|---|---|---|---|---|---|
| | | and concentration of the cross-linking agent | | | |
| | | EGDE, | PPGDE, | Acetone, | PEG-400, |
| | | mmol/g* | mmol/g* | % vol. | % vol. |
| Concentration | 4 | #1: 5 | #5: 5 | #9: 10 | #13: 5 |
| of RSF, % wt. | | #2: 30 | #6: 30 | #10: 20 | #14: 15 |
| | 5.5 | #3: 5 | #7: 5 | #11: 10 | #15: 5 |
| | | #4: 30 | #8: 30 | #12: 20 | #16: 15 |

The numerical data and size distribution curves (FIG. 9) show that an increase of injection pressure decreased the mean Feret diameter of particles, as well as narrowed the size distribution expressed as the span of recorded diameters within the cutoff range between the D10 and D90 cumulative values. For example, an increase of the injection pressure from 80 to 150 and then to 300 kPa resulted in decreased mean diameter of RSF (4% wt)/PPGDE (5 mmol/g)-derived RSFPs (Mixture #5, Table 1) from ca. 700 to 600 and 400 μm, respectively; at the same time, the D10/D90 diameter span narrowed from ca. 550 to 500 and 350 μm, respectively. These trends were also observed for the RSF/EGDE (5 mmol/g, Formulation #1, Table 1), RSF/acetone (10 & 20% vol., Formulations #9&10), and the RSF/PEG-400 (5&15% vol., Formulations #13&14) systems. However, concentrations of RSF above 5% wt had poor control over RSFPs size distribution. Reactive systems with 5 and 30 mmol/g of EGDE (Formulations #3&4) demonstrated a broadening of the D10/D90 particle size distributions by ca. 5-35% with increased injection pressure from 150 to 300 kPa, respectively. A similar tendency was observed for the RSF/pPGDE (Formulation #7) and the RSF/acetone (Formulations #11&12), despite a decrease in mean particle size. Typically, the shear viscosities of the 5.5% wt. RSF systems were 2-8 times lower than that of the 4% wt. systems (FIG. 9), which for the cryo-granulation approach may reflect the preferred viscosity range from 0.5 to 4 P to enable efficient control over the mean and minimum particle diameter, and the RSFPs size distribution. The most uniform sized distribution profiles were for the RSF (4% wt)/acetone (20% vol) Formulation #10; cryo-injections at either 150 or 300 kPa produced consistent D10/D90 sizes as narrow as ca. 170-250 μm, and the mean particle sizes of ca. 500 μm. The smallest particles with Feret diameter of ca. 150-200 μm were fabricated from the RSF/EGDE (Formulations #2, 3, 4) or RSF/pPGDE (Formulation #5) systems, while RSF/PEG-400 systems (Formulation #14) yielded the largest particles, with diameters of ca. 900-1,000 μm. The efficiency of the current cryo-granulation approach was confirmed gravimetrically by sieving; up to 90% wt. of the starting aqueous silk could be converted into the RSFPs with mean particle sizes between 200-900 μm. The additional sieve screening of the RSFPs fabricated from the RSF/EGDE or RSF/pPGDE mixtures estimated between 70 and 90% wt. of the obtained beads sized in range from 800 to 160 μm, which corresponded to ca. 0.6-3.6 grams of RSFPs per minute of cryogranulation with a liquid feed consumption rate around 20-30 mL/min depending on the injection pressure. Cryogranulation from the RSF/acetone mixtures resulted in higher yields, 80-95% wt. sized ca. 200-800 μm. We envision these diameters potentially being used for the production of large batches of polymeric meso-particles, followed by more precise size refinement (i.e. via sieve screening). The cryogranulation setup described above allowed processing of nearly 20-30 mL/min of the starting liquefied silk and produced between ca. 1 to 4.0 g/min of silk particles, surpassing microfluidic routes which process from 0.1 to 3.0 mL/min of liquefied silk. Thus, the cryogranulation method was an efficient alternative to microfluidic and emulsification technologies, with large monodisperse silk spheres.

The injection pressure and the composition of the starting RSF/cross-linker systems controlled the morphologies of the meso-RSFPs formed through the high-shear self-assembly. The non-solvent dispersion coupled with internal cryogelation generated the microporous inner morphology and wrinkled surface topology with the obtained meso-RSFPs. The textured surfaces covered the spherical RSFPs, which for most combinations of RSF/cross-linker revealed an interconnected microporous morphology of the cores (FIG. 10). The pore sizes ranged from 0.1 to 10 μm. This sponge-like morphology is a characteristic feature of the polymeric cryogels cross-linked either covalently or physically at sub-zero temperatures. The acetone- and PEG-400-treated RSFPs revealed similar microporous morphologies, with micropores in the 0.1-10 μm size. These pores in the RSFPs were significantly smaller compared to the large pores of ca. 50-150 μm generated in the prior macro-scale bulk cryogel samples, cross-linked through continuous −20° C. freeze-thaw aging following the addition of 5-30 mmol/g of EGDE or sonication. This observation may underline the direct dependence between the dimensions of the RSF-based cryo-construct and the sizes of the inner pores. The cryogenic treatment of reactive RSF-mixtures contained in macro-volumes produces larger pores, compared to the discrete micro- or meso-gels. Discrete RSFPs with collapsed structures were produced from the RSF-based systems containing 20% vol. acetone; the particles had a non-spherical “scrambled egg-shell” morphology with thin pore walls and rough edges and eroded surfaces (FIG. 10C).

No direct correlation between injection pressure and the surface topology of the cryo-RSFPs was observed. However, an increase of starting RSF concentration from 4 to 5.5% wt. yielded particles with more textured surfaces, including cracks, dimples or fractures. In particular, 5.5% wt. RSF with 10% vol. acetone resulted in coalesced beads, which slowed the dispersion step, quenched at the intermediate stage by the −50° C.-cooled coagulant. SEM micrographs did not reveal a decrease in RSFP size with increased RSF content. RSFPs stabilized with oligomeric PEG-400 and PPGDE displayed less static repulsion and thus more facile processability in the dry state, compared to the EGDE- and acetone-cured particles. RSFPs pre-encapsulated with DNR and DoxR displayed smooth surfaces with occasional micropores.

To evaluate the cytocompatibility of the RSFPs, we studied 30-day proliferation of RPTEC/TERT1 kidney and human normal fibroblast cell lines. Cell proliferation on the particles was characterized by confocal imaging. EGDE- and PPGDE-stabilized constructs supported cell growth over one month (FIG. 11).

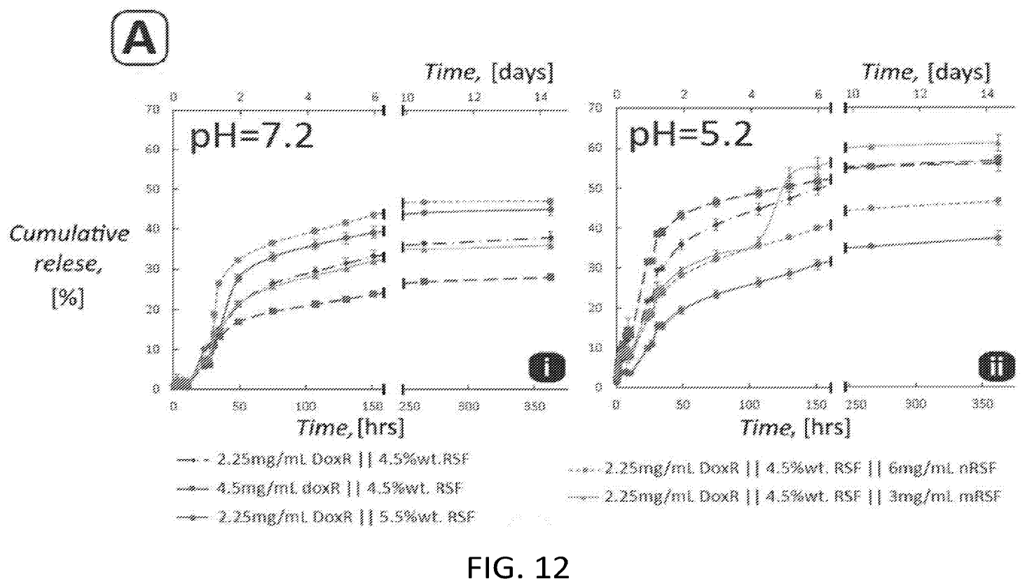

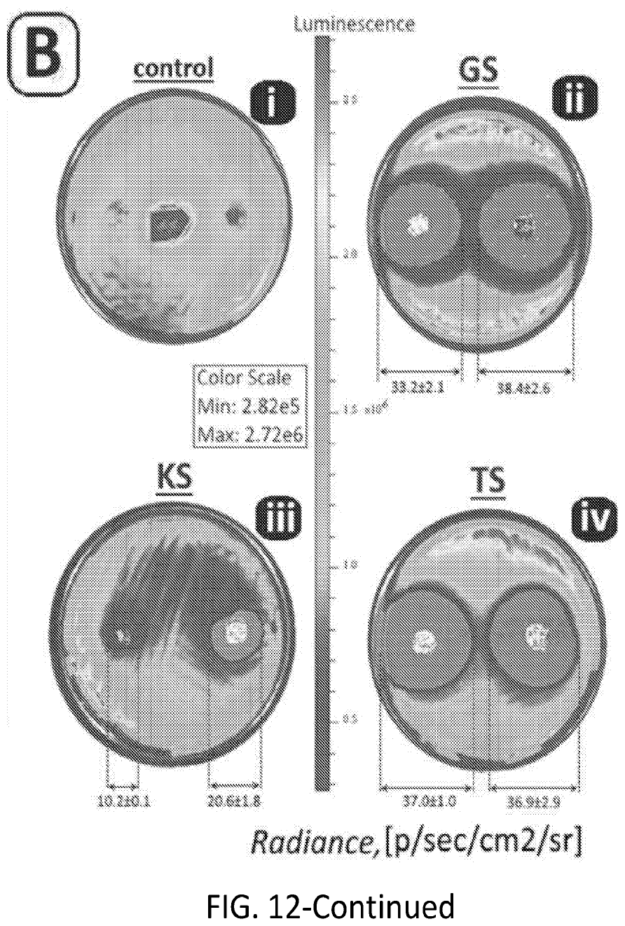

The affinity of silk towards anthracyclines provided prolonged release of DoxR, with faster release achieved at low pH levels. We obtained DoxR-encapsulated RSFPs with varying starting concentrations of RSF (4 and 5.5% wt.) and DoxR (2.25 and 4.5 mg/mL) to trace the impact of the drug/biopolymer ratio on pH-dependent pharmacokinetics. We assumed that the entire dosage of the drug was encapsulated within the resulting RSFPs as DoxR and the model antibiotics were poorly soluble in hexanes and had limited elution during the DEB cryo-fabrication step. The DoxR release from RSFPs was pH-dependent; 10-20% higher at pH 5.2, compared to the release profiles obtained at the pH 7.2. After 14 days, the total amount of DoxR released at pH 7.2 was 28-46%, depending on the starting formulation, whereas the cumulative release at pH 5.2 ranged from 38-62% (FIG. 12A). The first 3-4 days of DoxR elution were characterized by a burst release, reaching ca. 45% at pH 5.2, and ca. 38% at pH 7.2. Following the burst-release, consistent elution of DoxR at ca. 1-4%/day was established, which corresponded to a daily release of 0.1-0.21 mg/mL (from the 100 mg batch of particles). Interestingly, the lowest level of release at pH 7.2 (ca. 26%) was detected for RSFPs encapsulated with 4.5 mg/mL of DoxR (FIG. 12A, curve 2), compared to the RSFPs loaded with half the amount (2.25 mg/mL, FIG. 12A, curve 1) of drug. Solubility of DoxR increases with decreased pH and the 4.5 mg/mL-loaded particles demonstrated a nearly doubled cumulative release at pH 5.2. A reverse effect of the acid conditions was demonstrated for the release form RSFPs obtained from the concentrated (5.5% wt.) RSF systems; nearly 45% release of DoxR at pH 7.2 (FIG. 12A) was observed, and this decreased by ca. 10% (to 35%) at pH 5.2 (FIG. 12A). This interplay between concentration of dry RSF and eluting medium pH was observed for the silk DEBs modified with nano-RSF; providing the same amount of RSF in the starting mixture (4.5% wt.) RSFPs obtained with 6 mg/mL n-RSF (FIG. 12A) had the cumulative release of nearly 42% at pH 7.2, comparable to the release form RSFPs containing 5.5% wt. RSF (FIG. 12A). This was 10% higher compared to the release rates achieved by RSFPs fabricated using only 3 mg/mL of n-RSF (FIG. 12A).

Susceptibility tests were used to confirm post-processing antimicrobial activity of cryoencapsulated antibiotics, including tobramycin sulfate (TS), gentamicin sulfate (GS) and kanamycin sulfate (KS). The antibiotic function was preserved, evaluated by bacterial zones of inhibition (FIG. 12B) established on XEN29 cultures after 12 hours, as opposed to the negative controls of blank RSFPs (FIG. 12B, i). The diameter of the inhibition zones around the GS-loaded particles was 33.2±2.1 and around 38.4±2.6 mm in case of the 10 and 20 mg/mL pre-encapsulation concentrations (FIG. 12B, ii). Similarly sized inhibition zones were characteristic for TS-loaded RSFPs, measured at 37±1.0 mm for both 10 and 20 mg/mL pre-loaded particles (FIG. 12B, iv). Lower release efficiency was shown for the RSFPs loaded with either 10 or 20 mg/mL KS—(FIG. 12B, iii), which generated inhibition zones, respectively of around 21.3±1.8 and 10.2±0.1 mm after 48 hours. Overall, the results of the Kirby-Bauer assay demonstrated good retention of function of the antibiotics during the cry-granulation procedures.

In summary, forceful injections of RSF/cross-linker/drug compositions into water immiscible hexanes were used for production of silk beads with diameters ranging from 300 to 1,000 μm. Cryo-conditioning of the injection step by cooling the coagulation bath to ca. −65° C. arrested the shape and surface morphology of the droplets, originated from the spontaneous self-assembly after contact of emulsified RSF with the non-solvent phase. Additions of either diepoxide ethers, acetone, or polyethylene glycol played a dual function by emulsifying the reactive system enabling droplet formation and facilitating the process of either chemical (in the case of diepoxide ethers) or non-covalent internal cryogelation of silk. The freeze-thawed RSFPs had a microporous morphology resulting from ice templating which accompanied cryogelation, with pore sizes from ca. 0.1 to 10 μm in diameter. Mean diameter and width of the RSFPs distribution could be controlled by adjusting the bollus injection pressure from 80-300 kPa and by varying the viscosities of the starting reactive colloids. Injection pressures higher than 200 kPa favored the self-assembly of smaller particles of ca. 300-500 μm, while injections at lower than 100 kPa yielded the silk beads as large as 1,000 μm. The RSFPs obtained from the RSF/acetone and RSF/PPGDE mixtures had spherical morphologies with solid outer shells and spongy cores, while particles obtained with additions of EGDE- and PEG-400 had rough surfaces and prolonged or angular shapes. Drug-loaded particles demonstrated retention of bioactivity.

The present disclosure has described one or more preferred embodiments, and it should be appreciated that many equivalents, alternatives, variations, and modifications, aside from those expressly stated, are possible and within the scope of the invention.

We claim:

1. A method of making drug-eluting particles, the method comprising:
injecting a mixture into a super-cooled fluid, the mixture comprising regenerated silk fibroin and at least one medicament, wherein the super-cooled fluid has a temperature of less than −40 degrees Celsius.

2. A method of making drug-eluting particles, the method comprising:
injecting a mixture into a super-cooled fluid, the mixture comprising regenerated silk fibroin and at least one medicament, wherein the super-cooled fluid consists essentially of butane, pentane, hexane, heptane, octane, nonane, decane, or a mixture thereof.

3. The method of claim 1 or 2, the method further comprising
incubating the drug-eluting particles in the super-cooled fluid to promote cryogelation.

4. The method of claim 3, wherein the drug particles are incubated for between 5 and 15 minutes.

5. The method of any one of the preceding claims, the method further comprising:
extracting the drug eluting particles from the super-cooled fluid;
further incubating the drug eluting particles; and
lyophilizing the drug eluting particles.

6. The method of claim 5, wherein the drug eluting particles are further incubated at a temperature between −25 degrees Celsius and −15 degrees Celsius for at least 10 hours.

7. The method of claim 5 or 6, wherein the drug eluting particles are lyophilized at a pressure below 0.01 kilopascal.

8. The method of any one of the preceding claims, wherein the weight percent of silk fibroin in the mixture is between 3 and 6 percent.

9. The method of any one of the preceding claims, wherein the super-cooled fluid comprises hexane.

10. The method of any one of the preceding claims, wherein the temperature of the super-cooled fluid is between −60 degrees Celsius and −65 degrees Celsius.

11. The method of any one of the preceding claims, wherein the mixture further comprises at least one cross-linking agent.

12. The method of claim 11, wherein the at least one cross-linking agent is selected from the group consisting of ethylene glycol diglycidyl ether, poly(propyleneglycol)dyglycidyl ether, acetone, polyethylene glycol (PEG-400), or mixtures thereof.

13. The method of claim 11 or 12, the method further comprising:
mixing the cross-linking agent with an aqueous solution of the at least one medicament and the silk fibroin to form the mixture, wherein this step occurs prior to the injection of the mixture.

14. The method of claim 13, wherein the aqueous solution comprises a basic buffer.

15. The method of claim 13 or 14, wherein the mixing comprises vortexing the crosslinking agent and the aqueous solution.

16. The method of any one of the preceding claims, wherein the mixture is injected using a needle positioned above the surface of the super-cooled fluid.

17. The method of any one of the preceding claims, wherein the mixture is injected using a needle having an interior diameter between 0.4 and 0.5 millimeters.

18. The method of any one of the preceding claims, wherein the mixture is injected at a pressure between 80 and 300 kilopascals.

19. The method of any one of the preceding claims, wherein the medicament is an antibiotic.

20. The method of claim 19, wherein the antibiotic is selected from the group consisting of doxorubicin chloride, gentamicin sulfate, tobramycin sulfate, and kanamycin sulfate.

21. The method of claim any one of the preceding claims, wherein the mixture has a shear viscosity below 4 poise.

22. A method of treating a subject, the method comprising:
administering a drug-eluting particle made by the method of any one of the preceding claims to the subject.

23. The method of claim 22, wherein the drug-eluting particles are administered via an injectable drug delivery system.

24. A regenerated silk fibroin particle for drug elution prepared by the method of any one of claims 1 to 21.

25. The regenerated silk fibroin particle of claim 24, wherein the average diameter of the particles is between 300 and 800 μm.

26. The regenerated silk fibroin particle of claim 24 or 25, wherein the average pore size of the particles is between 0.1 to 10 μm.

27. The regenerated silk fibroin particle of any one of the preceding claims, wherein the particle has pores of a sufficient size to release the medicament.

28. The regenerated silk fibroin particle of any one of the preceding claims, wherein the particle is biodegradable.

29. A system for making drug-eluting particles, the system comprising:
a coagulation container system configured to retain a fluid and to maintain a temperature below −40 degrees Celsius for the fluid retained therein;
a super-cooled fluid located within the coagulation container system;
a source of a mixture comprising at least one medicament and regenerated silk fibroin, the mixture optionally comprising a cross-linking agent; and
an injection unit positioned above a surface of the super-cooled fluid and configured to inject the mixture into the super-cooled fluid.

30. The system of claim 29, wherein the injection unit is configured to continuously provide a uniform pressure to introduce the mixture.

31. The system of any one of the preceding claims, wherein the coagulation container system further comprises an extraction system configured to assist in the removal of the particles.

32. The system of any one of the preceding claims, the system further comprising a processor coupled to the system and configured to control one or more of the following operational parameters:
the temperature of the coagulation container system and/or the super-cooled fluid;
an injection pressure of the injection unit;
an injection gage of the injection unit;
a medicament concentration of the mixture;
a regenerated silk fibroin concentration of the mixture; or
a cross-linking agent concentration of the mixture.

33. A system for making drug-eluting particles, the system comprising one or more components described herein for the automatic execution of the method of any one of claims 1 to 21.
